(19) United States
(12) Patent Application Publication
(10) Pub. No.: US 2013/0024005 A1
(43) Pub. Date: Jan. 24, 2013

(54) COMPOSITE IMPLANT

(71) Applicant: Hilton Becker, Boca Raton, FL (US)

(72) Inventor: Hilton Becker, Boca Raton, FL (US)

(21) Appl. No.: 13/534,441

(22) Filed: Jun. 27, 2012

Related U.S. Application Data
(60) Provisional application No. 61/509,369, filed on Jul. 19, 2011.

(51) Int. Cl. A61F 2/02

(52) U.S. Cl. 623/23.72

(57) ABSTRACT

An implant includes a biocompatible framework material and a biologically-active material. The biologically-active material is embedded in the biocompatible framework material, and a portion of the biologically-active material is exposed to the outside of the implant.

### CROSS REFERENCE TO RELATED APPLICATIONS

This application claims the benefit of an earlier filing date from U.S. Provisional Application Ser. No. 61/509,369 filed Jul. 19, 2011, the entire disclosure of which is incorporated herein by reference in its entirety.

### BACKGROUND

An implant may be introduced into a human body to replace, support, or enhance a structure within the body. When a foreign body is introduced into a human body as an implant, it may be encapsulated by scar tissue, forming a capsule. Scar tissue includes the protein collagen, which in scar tissue may be cross-linked and aligned in a single direction. This may cause scar tissue to have relatively lower functional quality than collagen in normal, non-scar tissue. Thus, an implant surrounded by a scar tissue capsule may not be well integrated to the rest of the biological structures within the body, and have an undesirably low level of bio-integration.

There have been various attempts to improve bio-integration of implants. Surface texturing of an implant made of silicone creates a porous, sponge-like surface. Living body tissue may grow into the cavities to fix the implant to the body. However, a living body may react to synthetic material such as silicone by forming a capsule of scar tissue around it (as an oyster forms a pearl around a grain of sand). A non-living tissue implanted in the human body that becomes encapsulated with scar tissue may have several detrimental effects. Also, if a non-living tissue is exposed through the skin, it may become infected.

Also, materials such as hyaluronic acid, collagen, and polylactic acid may be applied to the surface of an implant. Living tissue will grow into these biologically-active materials, encouraging bio-integration of the implant in the body. However, these biologically-active materials may be absorbed into the blood supply within living tissue that grows near the implant. The absorbed materials may leave an undesirable textured surface around the implant.

### BRIEF DESCRIPTION

The above and other deficiencies of the prior art are overcome by, in an embodiment, a composite implant that has improved bio-integration.

In another embodiment, a composite implant comprises a silicone and a biologically active material that has improved bio-integration.

In a further embodiment, an implant comprises a biocompatible framework material and a biologically-active material, wherein the biologically-active material is embedded in the biocompatible framework material, and a portion of the biologically-active material is exposed to the outside of the implant.

It is to be understood that both the foregoing general description and the following detailed description are exemplary and explanatory and are intended to provide further explanation of the invention as claimed.

### BRIEF DESCRIPTION OF THE DRAWINGS

The following descriptions should not be considered limiting in any way. With reference to the accompanying drawings, like elements are numbered alike:

FIG. 1 is an image of a hair;

FIG. 2 is a cross-sectional view of the hair shown in FIG. 1;

FIG. 3 shows a cross-sectional view of a silicone implant;

FIG. 4 shows the silicone implant of FIG. 3 after being implanted in a living body;

FIG. 5 shows a cross-sectional view of a surface-textured silicone implant;

FIG. 6 shows the surface-textured silicone implant of FIG. 5 after being implanted in a living body;

FIG. 7 shows a cross-sectional view of a surface-textured silicone implant including a biologically-active matrix material;

FIG. 8 shows the surface-textured silicone implant including a biologically-active matrix material of FIG. 7 after being implanted in a living body;

FIG. 9 shows a cross-sectional view of a surface-textured silicone implant including a biologically-active matrix material;

FIG. 10 shows the surface-textured silicone implant including a biologically-active matrix material of FIG. 9 after being implanted in a living body;

FIG. 11 shows a cross-sectional view of a silicone implant with strands of biologically-active matrix material disposed therein according to an exemplary embodiment;

FIG. 12 shows a bottom view of the silicone implant shown in FIG. 11;

FIG. 13 shows a top view of the silicone implant shown in FIG. 11;

FIG. 14 and FIG. 15 show a cross-sectional view of a silicone implant in accordance with an exemplary embodiment and an inset view, respectively;

FIG. 16 shows a cross-sectional view of a silicone implant with a biologically-active matrix material according to an exemplary embodiment; and

FIG. 17 shows a cross-sectional view of a silicone implant with granules of biologically-active matrix material according to an exemplary embodiment.

### DETAILED DESCRIPTION OF THE ILLUSTRATED EMBODIMENTS

A detailed description of one or more embodiments is presented herein by way of exemplification and not limitation.

Human teeth, nails, and hair have similar structures with each other, in that they are formed from living tissue within the body, and then through a transitional structure become non-living but remain integrally attached to the living tissue of the body. This property improves bio-integration and decreases the ability of teeth, nails, and hair to detach from the body. The base of these structures is living vascularized cellular (i.e., biological) material, while the distal end is non-living, non-vascularized acellular material.

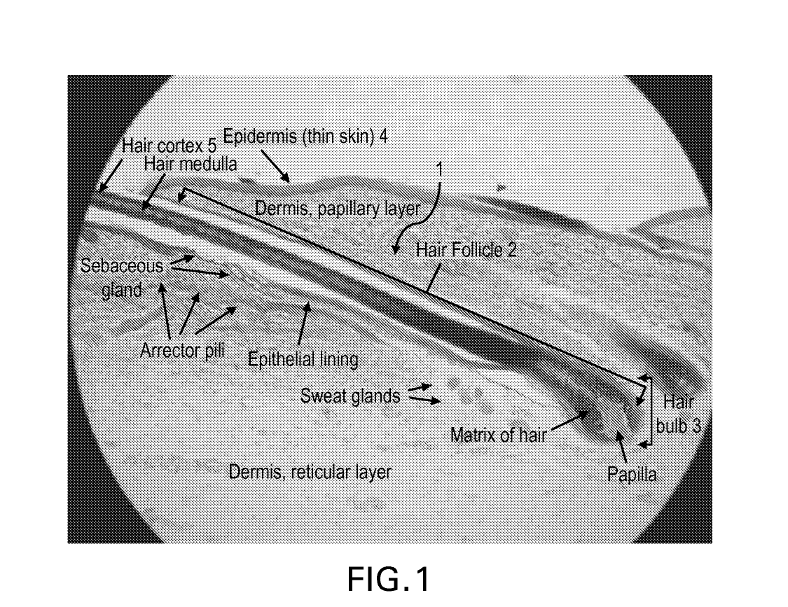

FIG. 1 shows an image of a hair 1. A follicle 2 and hair bulb 3 are regions beneath the skin 4 and within the body which grow the hair 1. The follicle 2 is the living portion of the hair 1, and the hair bulb 3 contains cells that produce a hair shaft 5. The end of the follicle 2 and the hair bulb 3 show an area where living tissue and blood supply connect to the hair 1. The hair shaft 5 is the visible portion of the hair 1 that extends beyond the skin 4 and body. The hair shaft 5 exhibits no biochemical activity and is considered non-living. A portion of the hair 1 attached to living tissue through the transitional structure may not become infected although it is exposed outside the body.

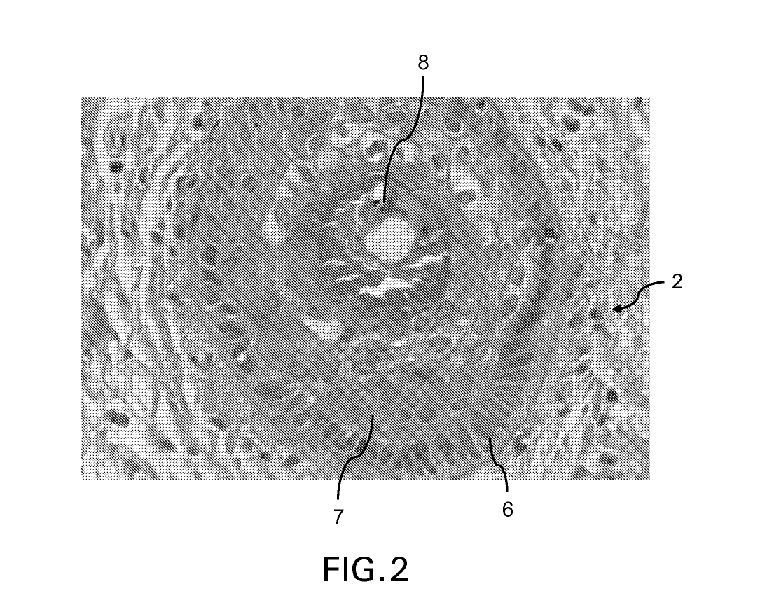

FIG. 2 shows a cross-sectional view of the hair follicle 2. The outer-most layer of hair is the cuticle 6, which is several layers of flat, thin cells that overlap each other. The next inner layer is the cortex 7 that contains protein in rod-like structures. The inner-most layer is the medulla 8, which is a disorganized area of cells at the follicle's center.

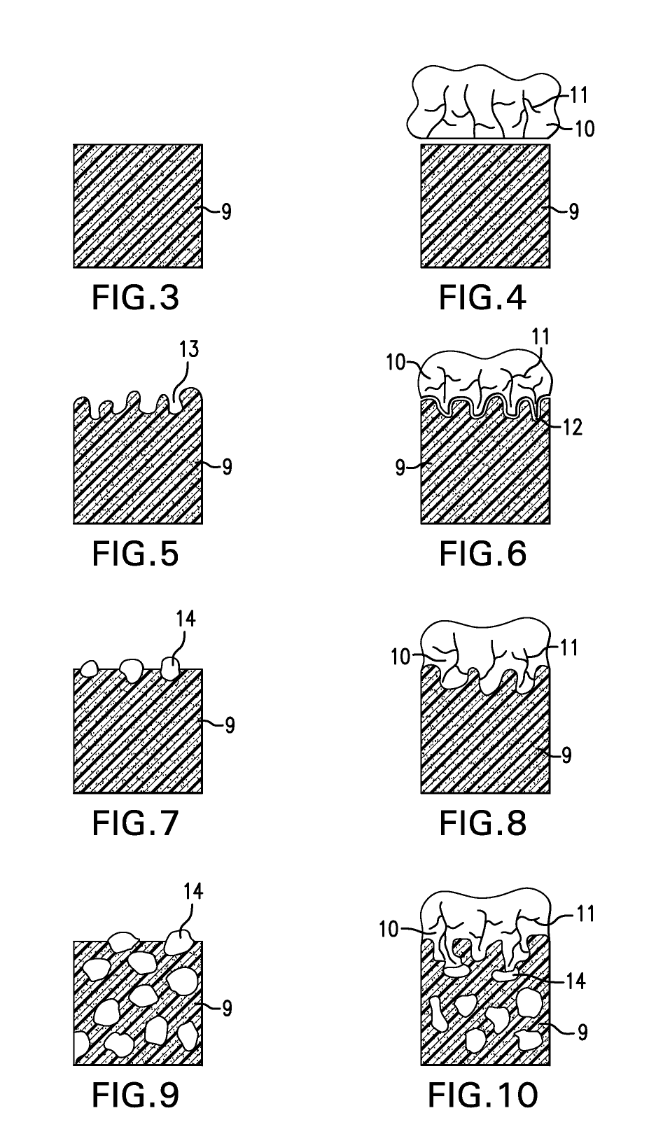

FIG. 3 through FIG. 10 shows various cross-sectional views of examples of implants and subsequent bio-integration of the implants. For simplicity, only bio-integration of the top surface of each implant is shown. However, bio-integration may occur at other surfaces of the implant. Specifically, FIG. 3 shows a silicone implant 9, and FIG. 4 shows the silicone implant 9 after being implanted in a living body and bio-integration has occurred. An embodiment includes an implant framework material made of silicone. However, other biocompatible materials may be used, such as polytetrafluoroethylene (Teflon), polyethylene, polypropylene, nylon, polytetrafluouroethylene (PTFE), calcium, coral, acellular bone, and the like. Living tissue 10 and blood vessels 11 grow near the surface of the silicone implant 9; however, a scar tissue capsule 12 can form between the living tissue 10 and blood vessels 11 and the silicone implant 9, preventing substantial bio-integration.

FIG. 5 shows a surface-textured silicone implant 9, and FIG. 6 shows the silicone implant 9 after being implanted in a living body. Living tissue 10 and blood vessels 11 grow near the surface of the silicone implant 9; however, a scar tissue capsule 12 may form between the living tissue 10 and blood vessels 11 and the silicone implant 9, preventing substantial bio-integration, similar to the silicone implant shown in FIG. 4. Since the surface of this silicone implant has a textured region 13, the implant may be better attached to the living tissue 10 and blood vessels 11 within the living body, relative to the silicone implant 9 without surface texturing.

FIG. 7 shows a surface-textured silicone implant 9 including granules 14 of biologically-active matrix material, and FIG. 8 shows the silicone implant 9 after being implanted in a living body and bio-integration occurs. According to an embodiment, the biologically-active matrix material can include hyaluronic acid, collagen, and polylactic acid and can include such commercially-available products as Gore® BioA®, LifeCell™ Alloderm®, Bard™ Allomax™, and LifeCell™ Stratus®. The biologically-active matrix material can also be collagen containing tissue such as acellular dermis obtained from human, porcine, or bovine skin. The biologically-active matrix material can also be a synthetic material such as Gore® Bio-A®, vicryl, polyglycolic acid, trimethylene carbonate, and the like. As living tissue 10 and blood vessels 11 grow near the surface of the silicone implant 9, blood vessels 11 grow into the granules 14. When blood vessels 11 grow into the granules 14, the granules may be consumed entirely by the blood vessels 11, allowing living tissue 10 and blood vessels 11 to occupy the space formerly occupied by the granules 14 (the biologically-active matrix material is therefore referred to herein as “bio-integratable”). Thus, bio-integration of the silicone implant 9 is increased relative to the silicone implants shown in FIG. 4 and FIG. 6. However, as shown in FIG. 8, since the granules 14 are only disposed in the silicone implant 9 near the surface thereof and have a relatively small depth into the silicone implant 9, bio-integration may be limited to the depth that the granules 14 penetrate into the silicone implant 9.

FIG. 9 shows a surface-textured silicone implant 9 including granules 14 of biologically-active matrix material, and FIG. 10 shows that silicone implant 9 after being implanted in a living body and bio-integration occurs. Similar to the implant shown in FIG. 7, as living tissue 10 and blood vessels 11 grow near the surface of the silicone implant 9, blood vessels 11 grow into the granules 14. Thus, bio-integration of the silicone implant 9 having biologically-active matrix material is increased relative to the silicone implants shown in FIG. 4 and FIG. 6. Since the granules 14 are disposed throughout the silicone implant 9, as FIG. 10 shows, blood vessels 11 and living tissue 10 can grow further into the implant than as shown in FIG. 8. However, since the biologically-active matrix material shown in FIG. 7 through FIG. 10 is in granule form, ingrowth of blood vessels 11 and living tissue 10 can be uneven or incomplete, resulting in inadequate bio-integration.

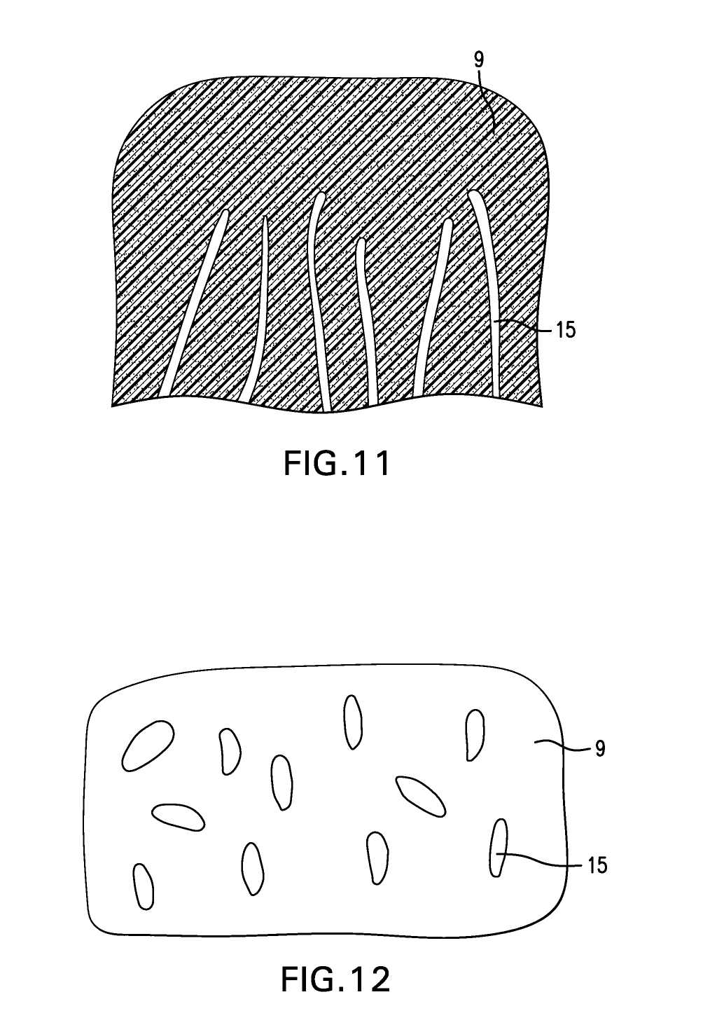

FIG. 11 shows a cross-sectional view of a silicone implant 9 with strands 15 of biologically-active matrix material disposed therein according to an exemplary embodiment. Each strand 15 of biologically-active matrix material extends along an extending direction into the silicone implant 9 in order to allow sufficient bio-integration with the living body into which the silicone implant 9 is implanted. The strands 15 can extend at least half the length or thickness of the silicone implant 9 along the extending direction of the strands 15. As shown in FIG. 11, the length of the strands 15 along the extending direction can be greater than the width of the strands 15. The strands 15 of biologically-active matrix material can be arranged in any fashion, and in an embodiment the strands 15 extend into the silicone implant 9.

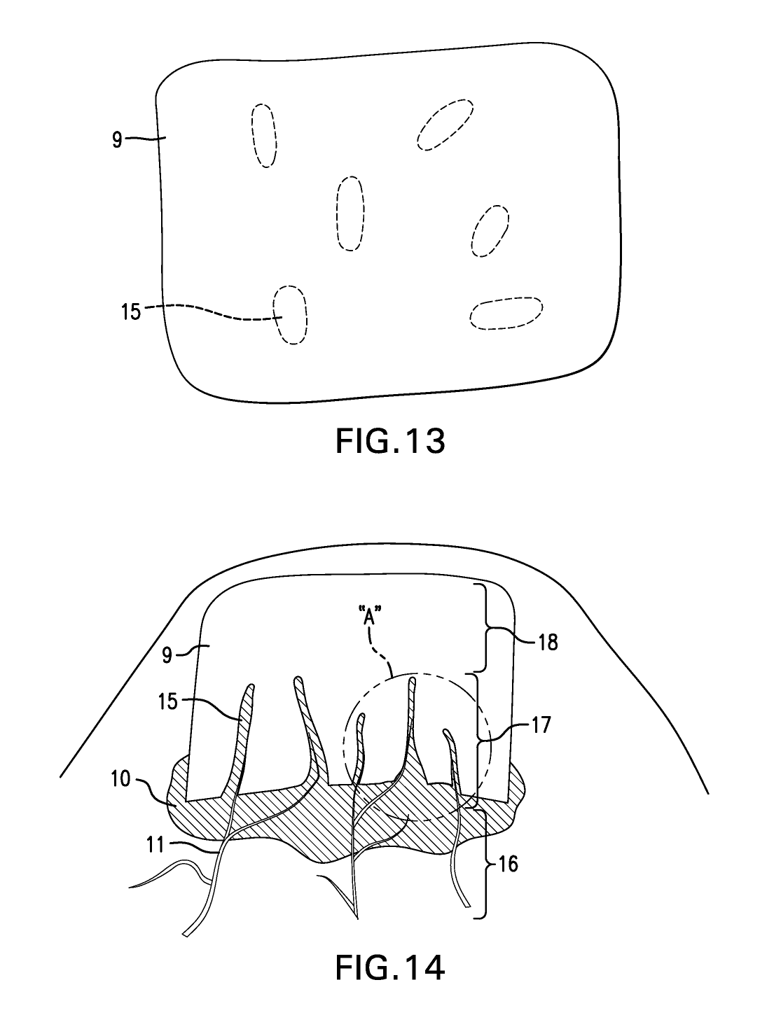

FIG. 12 shows a bottom view of the silicone implant 9 shown in FIG. 11. Each strand 15 of biologically-active matrix material according to the present exemplary embodiment has an oval or rectangular shape but may have any shape. Each strand 15 of biologically-active matrix material is surrounded by silicone. Because each strand 15 is surrounded by silicone, the structural integrity of the silicone implant 9 can be improved. Alternatively, strands 15 of biologically-active matrix material can cross each other within the silicone implant 9 to form a mesh. In this case, the strands 15 of biologically-active matrix material can contact each other in the mesh. FIG. 13 shows a top view of the silicone implant 9 shown in FIG. 11. As indicated by the dotted outline of each strand 15 of biologically-active matrix material, the strands 15 do not penetrate the upper surface of the silicone implant 9.

There are any number of alternative arrangements of the strands 15 in the silicone implant 9 besides that shown in the present exemplary embodiment. For example, the strands can be arranged side to side, top to bottom, side to top, side to bottom, etc., or any combination of these arrangements. The strands 15 may not be straight but can have a curved shape. Further, strands 15 can be grown in by blood vessels 11 and living tissue 10 from more than one side of the silicone implant 9.

FIG. 14 shows a cross-sectional view of the silicone implant 9 in accordance with the present exemplary embodiment. The silicone implant 9 is embedded within a living body, and bio-integration has occurred. As seen in FIG. 14, the silicone implant 9 is surrounded by living tissue 10 and blood vessels 11. Scar tissue (not shown) can form between the living body and the silicone implant 9 on the sides of the silicone implant 9 exposed to the living body. Bio-integration is shown by blood vessels 11 and living tissue 10 penetrating into the silicone implant 9 via the strands 15 of biologically-active matrix material. The blood vessels 11 and living tissue 10 that have penetrated into the silicone implant 9 are referred to as ‘secondary’ blood vessels and ‘secondary’ living tissue. Secondary blood vessels 11 and secondary living tissue 10 grow into, dissolve, and absorb the strands 15 of biologically-active matrix material and fill the holes in the silicone implant 9 created by the strands 15 biologically-active matrix material. Thus, the living body remodels the biologically-active matrix material.

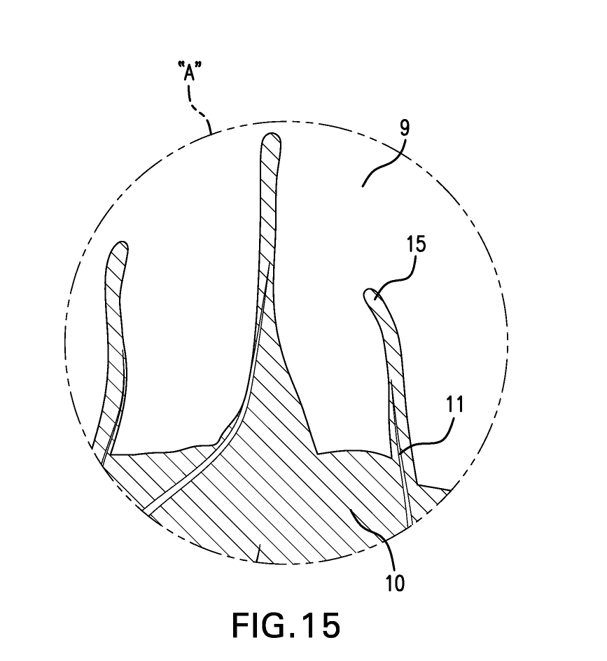

The process of bio-integration creates three connected regions in the area of the living body where the silicone implant 9 has been implanted. The first region is a living zone 16, which is in the area of the living body where the blood vessels 11 and living tissue 10 grow originally. Next is the transitional zone 17, which contains secondary blood vessels 11 and secondary living tissue 10 that has absorbed the biologically-active matrix material strands 15 and therefore extends into the silicone implant 9. The biologically-active matrix material strands 15 are at least partially bio-integrated in the transitional zone 17. The transitional zone 17 is shown in greater detail in FIG. 15. The last region is the non-living zone 18, which either is solely made of the silicone implant 9 or may contain some part of the biologically-active matrix material strands 15 that have not been bio-integrated.

The three regions in the area of the living body where the silicone implant 9 has been implanted create a junctional structure. As the secondary blood vessels 11 and secondary living tissue 10 penetrate further into the silicone implant 9, they can become smaller and less able to penetrate. However, since the strands 15 of biologically-active matrix material extend into the silicone implant 9 a certain distance, the silicone implant 9 according to the present exemplary embodiment exhibits improved adhesion, strength, and durability once bio-integration has occurred. A scaffold created by the strands 15 due to the junctional structure holds the silicone implant 9 to the blood vessels 11 and the living tissue 10.

By creating a transitional zone, a scar tissue capsule may not form between the silicone implant 9 and the blood vessels 11 and the living tissue 10, and exteriorization of the implant can be facilitated. Thus, dental implants and fixation devices for external prostheses such as ears, noses, and the like can be more easily formed compared with other implants. The transitional implant can also find application in buried prostheses such as joint, facial, chin, and skull implants. In these implants, fixation to the transitional zone can prevent bone resorption commonly seen with conventional silicone implants.

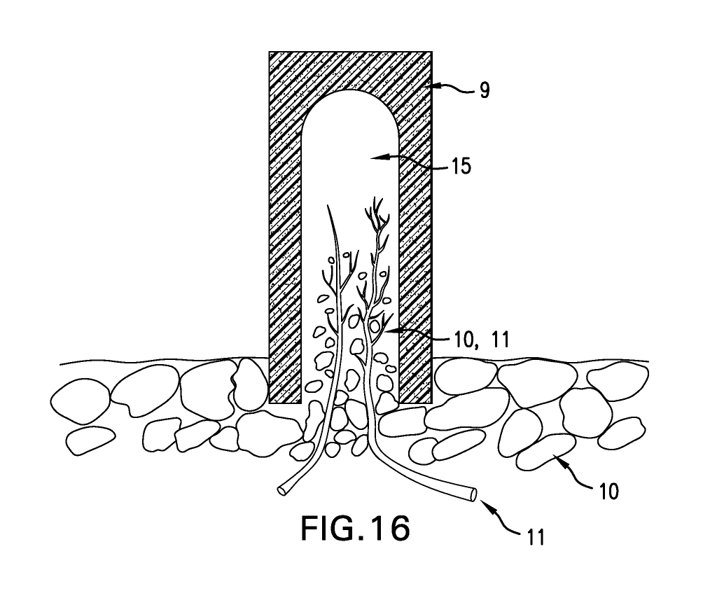

FIG. 16 shows a sectional view of a silicone implant 9 with a biologically-active matrix material according to an exemplary embodiment. Similarly to the exemplary embodiment described above with respect to FIG. 11 through FIG. 15, the silicone implant 9 contains a strand 15 of biologically-active matrix material, such as collagen. In the present exemplary embodiment, however, the implant contains a single strand 15 rather than a plurality of strands 15 of biologically-active matrix material. The present exemplary embodiment has the same three regions as described above, with blood vessels 11 and living tissue 10 from the living zone 16 growing into the strands 15 of biologically-active matrix material in the transitional zone 17, and the top portion of the silicone implant 9 being the non-living zone 18. FIG. 16 shows blood vessels 11 in the transitional zone 17 become smaller as they grow further into the strands 15 of biologically-active matrix material, finally stopping before the non-living zone 18. Further, the alternative arrangement of strands described above with respect to FIG. 11 through FIG. 13 can be used in the present exemplary embodiment.

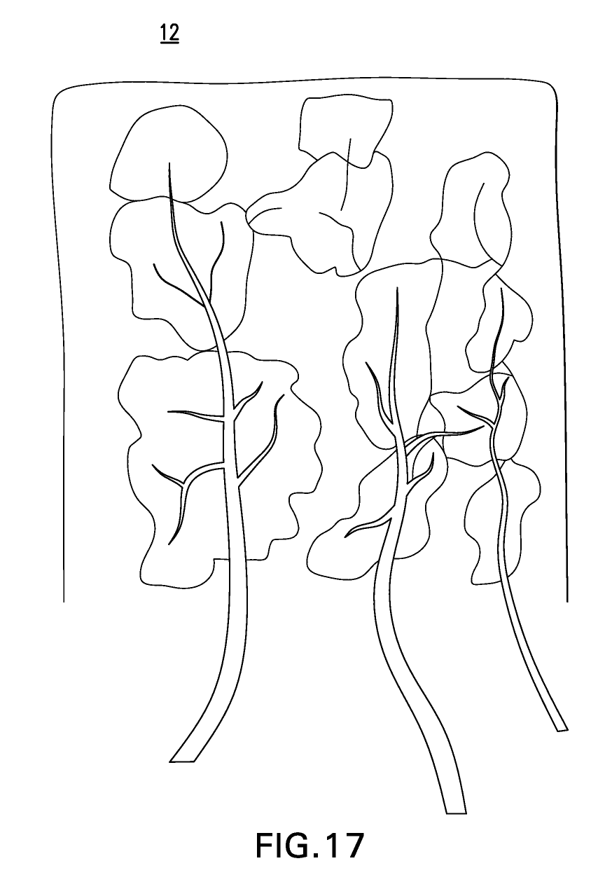

FIG. 17 shows a cross-sectional view of a silicone implant 9 with granules 14 of biologically-active matrix material according to an exemplary embodiment. The present exemplary embodiment is similar to those shown in FIG. 11 through FIG. 15, except that instead of strands 15, granules 14 of biologically-active matrix material are used. The granules 14 are substantially in continuity in the present exemplary embodiment, so that the junctional structure can form, as described above. The granules 14 have a length that is about the same as a width thereof.

Exemplary embodiments show blood vessels 11 and living tissue 10 growing into the biologically-active matrix material from one side of the silicone implant 9. This structure can be suitable for implants where a smooth and non-bio-integrated surface is desired. However, more thorough bio-integration can be possible if the biologically-active matrix material is accessible to blood vessels and living tissue from multiple sides of the implant. According to an embodiment, the length of biologically-active matrix material in the implant can be arranged such that the junctional structure is created, to form a scaffold between the silicone and the blood vessels.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like. All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” It should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity). The conjunction “or” is used to link objects of a list or alternatives and is not disjunctive, rather the elements can be used separately or can be combined together under appropriate circumstances.

It will be understood that when an element or layer is referred to as being “on” or “connected to” another element or layer, it can be directly on or directly connected to the other element or layer, or intervening elements or layers may be present. In contrast, when an element is referred to as being “directly on” or “directly connected to” another element or layer, there are no intervening elements or layers present.

What is claimed is:

1. An implant, comprising:
a biocompatible framework material; and
a biologically-active material,
wherein the biologically-active material is embedded in the biocompatible framework material, and a portion of the biologically-active material is exposed to the outside of the implant.

2. The implant of claim 1, wherein the biologically-active material comprises a plurality of strands.

3. The implant of claim 2, wherein each strand comprises a length greater than a width along an extending direction, and the strand extends into the biocompatible framework material along the extending direction.

4. The implant of claim 2, wherein each strand is surrounded by the biocompatible framework material except for the portion exposed to the outside of the implant.

5. The implant of claim 2, wherein a first strand contacts a second strand within the implant.

6. The implant of claim 1, wherein the portion of the biologically-active material exposed to the outside of the implant is bio-integratable with a living body.

7. The implant of claim 6, wherein secondary living tissue and secondary blood vessels is to grow into, dissolve, and absorb the biologically-active material.

8. The implant of claim 6, wherein the implant comprises a transitional zone where the biologically-active material is to be partially bio-integrated.

9. The implant of claim 6, wherein the implant comprises a non-living zone that cannot bio-integrate with the living body, the non-living zone comprising the biocompatible framework material or the biologically-active material.

10. The implant of claim 6, wherein a distal end of the biologically-active matrix material opposite to the exposed portion is not bio-integratable with the living body.

11. The implant of claim 6, wherein a distal end of the biologically-active matrix material opposite to the exposed portion is bio-integratable with the living body.

12. The implant of claim 1, wherein the biologically-active matrix material comprises a plurality of granules, each granule comprising a length that is about the same as a width thereof.

13. The implant of claim 12, wherein at least two granules contact each other along a first direction of the implant, the first direction extending into a center portion of the implant.

14. The implant of claim 1, wherein only the biologically-active material is bio-integratable.
